(19) United States
(12) Patent Application Publication
(10) Pub. No.: US 2005/0163741 A1
(43) Pub. Date: Jul. 28, 2005

(54) Cosmetic preparation

(76) Inventor: Christina Zech, KAUFERING (DE)

Correspondence Address: BACHMAN & LAPOINTE, P.C., 900 CHAPEL STREET, SUITE 1201, NEW HAVEN, CT, 06510, US

(21) Appl. No.: 10/509,193

(22) PCT Filed: Mar. 6, 2003
(86) PCT No.: PCT/EP03/02327

(51) Int. Cl. A61K 7/06; A61K 7/11

(52) U.S. Cl. 424/70.16; 424/70.17

(57) ABSTRACT

An aqueous-based preparation for application to the skin or skin appendages, which contains a mixture of at least two aqueous dispersions of film-forming polymers of which one is a polyurethane-based polymer and the other is an acrylate-based polymer, and optionally conventional constituents, wherein the preparation is of a viscosity in the range of between 0.02 and 3.8 Pa.s measured at 25° C. and with 200 s⁻¹.

### BACKGROUND OF THE INVENTION

The invention concerns a preparation for application to the skin or skin appendages and the use thereof, a process for the production thereof and a device for the application thereof.

The preparation according to the invention can be used for decorative purposes, for making up the skin, the semi-mucous membranes or regions of the skin in the proximity of mucous membranes. It can also be applied to skin appendages. Thus there are products which can be applied to skin appendages such as hair, eyelashes and eyebrows or also the artificial reproductions thereof in order to impart desired properties thereto. It is known for keratin fibers to be covered with a coating which imparts fullness and volume, an attractive shape and/or also color. For example eyelashes can be provided with a colored coating, which emphasizes them, shapes them and possibly also makes them longer. It is further known for hair to be provided with a coating which smoothes the hair, gives volume thereto or possibly also colors individual strands.

Preparations for making up the skin or the semi-mucous membranes are generally known in the form of pencils, in the form of pastes which are cast into small metal pots, in the form of soft pastes or in the form of loose or pressed powders. Pencils or pastes are generally in water-free form as mixtures of vegetable, animal or synthetic oils, fats and waxes, in which there is dispersed a powder paste comprising pigments which are allowed for cosmetics, pearl gloss agents and fillers such as talc, kaolin or amorphous silicon dioxide. Loose or pressed powders generally comprise mixtures of pigments which are allowed for cosmetics, pearl gloss agents and fillers which can be put into a handleable or workable form by means of a binding agent on an oil base or in the form of an emulsion.

All those preparations suffer from the disadvantage that they only inadequately adhere to the location where they are applied and can be easily transferred onto other materials such as crockery, cutlery, glass, textiles or skin. That gives rise to the need for those preparations to be repeatedly applied. As oils spread on the skin and on semi-mucous membranes and in addition also involve differing spreading capability, constituents of those preparations, in particular pigments, can bleed out of lipsticks into the fine creases in the skin around the lips and thus produce structures which are a nuisance because they are unattractive. Those effects can be provoked or boosted by the sebum of the skin and by perspiration. Eye-shadows in powder form for example can migrate in particular into the creases in the eyelid due to the movement of the lids and due to the effect of sebum, and as a result can produce strip-shaped structures.

The attempt has been made since 1978 to produce wax-based cosmetic pencils, in particular those with leads which are cast into injectable cases, on pigmented wax mixtures which contained a marked proportion of volatile oils, preferably volatile silicone oils such as cyclomethicones or hydrocarbons such as isoparaffins or mixtures thereof. Those preparations were at times also improved in respect of their properties of use by additions of non-volatile silicones such as alkyldimethicone or phenyl trimethicone. Particularly when using micas coated with metal oxides, it was possible to achieve very soft and malleable and high-gloss preparations with very good adhesion capability, which were very well accepted on the market by the consumer.

A disadvantage in this respect however was the content of volatile constituents, which on the one hand required very good sealing of the pencil materials used such as casing, closure cap and end cap, and which on the other hand sometimes resulted in consumer complaints if such cosmetic pencils were stored open without a closure cap for a prolonged period of time because they then shrink because of evaporation of the volatile constituent and harden to the point of no longer being suitable for use. On the other hand female consumers felt water-free preparations based on oils, fats and waxes as forming an unpleasant film on the skin or on semi-mucous membranes, particularly if that preparation was applied in a relatively thick layer.

The attempt has therefore already been made to produce aqueous or aqueous-alcoholic preparations in the form of polymer-bearing dispersions which no longer contained any oils, fats or waxes, which were intended to no longer suffer from the above-discussed disadvantages and which formed elastic and more or less water-resistant films on the skin or on semi-mucous membranes. Thus for example G. A. Nowak, in “Die kosmetischen Präparate”, 1st edition, 1969, pages 588 and 589, describes various compositions of eyeliners and liquid makeup which are based on synthetic film-forming agents such as polyvinyl pyrrolidone (PVP) or carboxymethylcellulose or natural film-forming agents such as shellac, tragacanth or gum arabic. On page 441 G. A. Nowak describes lipsticks based on solvents such as ethyl alcohol or isopropanol which as film-forming agents contain ethyl cellulose, cellulose acetate, shellac, methyl abietate or PVP. Such preparations however have evidently not proved worthwhile in practice because of the content of solvents. EP-A 0 793 957 describes a composition which can be applied to the skin, the semi-mucous membranes or the membranes, which contains an aqueous dispersion of particles of a film-forming polymer which, on a carrier, forms a film of a hardness of less than 110 (determined in accordance with the standard NF-T-30-016). The examples each describe an eyeliner and a lip rouge based on polyurethane dispersions. Those compositions are intended to form a film which adheres well to the substrate and which is soft and flexible, which follows the movement of the skin and does not crack and which does not become detached. Those compositions are intended not to bleed on the skin and not to be transferred onto other materials.

In addition EP-A 1 010 418 discloses a wax-free gel composition which contains polyurethane polymers as film-forming agents. That composition is used as mascara and is intended to adhere to the eyelashes for longer than a day. To adjust the viscosity, preferably a pyrogenic silicic acid is used for those compositions, but the silicic acid, particularly when applied more heavily results in the material flaking off.

EP-A 1 249 225 also describes a wax-free gel composition which is said to be water-resistant and which is said to adhere for more than two days. That composition is intended to be used in particular as a coloring agent for hair, eyelashes and eyebrows and after application and drying is intended to form a smooth coating. It will be noted that there is the disadvantage here that the pyrogenic silicic acid contained in the composition severely thickens it, even when present in a relatively low level of concentration, and causes the dried film to appear dull. As in addition it is not hydrophobized, it promotes water absorption on the part of the film and thus reduces the water-resistance and thus its durability.

In addition U.S. Pat. No. 6,458,390 describes a makeup composition which contains iron oxide pigments and film-forming agents and which is said to adhere for a long time. It will be noted that, for this composition, it is necessary to use a specific silicone acrylate copolymer dissolved in a volatile solvent. In comparison the invention involves using an aqueous dispersion. Aqueous-based products can contain the constituents either in dissolved or dispersed form, in the latter case it is necessary to take care to ensure that, when constituents such as for example pigments are not dissolved, they do not settle with time and thus result in products which are unusable.

In addition with all these products there is the requirement that, after they have been applied, they remain in position and do not run, come off, flake away or bleed out. In the ideal situation the products should be resistant even when bathing or showering, that is to say upon coming into contact with water, possibly even in connection with shampoos, shower preparations or soap. Specifically for eyelash mascara it is desirable for it to remain on the eyelashes, even when it comes into contact with water. In addition it should not come off when rubbed and in particular should not color the area around the eye. In addition the coating should be such that it retains the constituents thereof so that portions thereof cannot bleed away therefrom and color the surrounding area.

Therefore the object of the present invention was to provide a preparation, in particular a cosmetic preparation, which can be applied for decorative purposes for makeup to the skin, to semi-mucous membranes and/or in the proximity of semi-mucous membranes. That is intended to denote in particular application to the skin of the face and/or the body, to the lips and to the eyelids and to lid edges. In particular lip rouge, blusher, makeup, eye-shadow, lipliner, eyeliner, concealer and body paints such as so-called temporary tattoos or Mehndi decoration are intended to be interpreted as preparations for makeup.

A further object of the invention was to provide a cosmetic preparation which can be applied to skin appendages and which adheres there for a prolonged period of time, for example more than 8 hours and in particular more than a day, remaining at the location at which it was applied, without being removed by water, perspiration or contact with articles, but which on the other hand can also be easily removed again. A further object of the invention was to provide a wax-free preparation which provides a durable and glossy coating. In addition the invention aimed to provide a preparation which can be easily applied and which enjoys a high level of storage stability.

### SUMMARY OF THE INVENTION

In accordance with the invention to attain that object a mixture is used comprising at least two aqueous dispersions of film-forming polymers, which leads to better adhesion and longer storage capability.

Accordingly, the aqueous-based preparation according to the invention for application to the skin or skin appendages contains a mixture of at least two aqueous dispersions of film-forming polymers of which one is a polyurethane-based polymer and the other is an acrylic-based polymer, and optionally conventional constituents. In a preferred embodiment which is particularly suitable for application to the skin appendages the preparation is of a viscosity in the range of between 0.02 and 3.8 Pa·s, measured at 25° C. and with 200 s⁻¹.

### DETAILED DESCRIPTION

The preparation according to the invention is in the form of an aqueous dispersion which after application dries to provide a water-resistant film and which enjoys excellent adhesion to the location at which it was applied. That film is elastic and stretchable and can therefore follow the movements of the skin and semi-mucous membranes or the hair. In addition, upon drying, it neither contracts nor does it shrink to a considerable degree so that it does not produce any feeling of pulling on the skin or the lips and also does not flake off. It also does not become cracked or grainy and it is so elastic and durable that it does not become detached from the edge of the application or even as a whole—rather it remains “like a second skin” at the location at which it was applied. Its constituents do not move, not even under the influence of sebum or perspiration, and its constituents also cannot be transferred onto other materials. In general the film already exhibits a high surface gloss and high reflection capability upon drying, but at the latest after complete drying. After complete drying the film is so stable that it is not detrimentally altered by a possible second application of the preparation. In no way may the dried film stick or give a sticky feel.

If the preparation according to the invention contains coloring agents, they must involve such grain sizes, surface properties and forms that the coloring agents do not detrimentally influence film formation although—so-to-speak as “foreign bodies”—they interfere with the formation of the film. The following can be mentioned as coloring agents which are suitable for cosmetic preparations: inorganic pigments, for example yellow, red or black iron oxides, ultramarine, chromium oxide green, chromium hydroxide green, carbon black and/or optionally organic pigments, lakes of organic coloring agents, metal powders in flake form, for example passivated aluminum, brass, bronze, copper, silver or gold, mica, mica coated with metal oxides, for example with titanium dioxide, iron oxides, chromium oxide, chromium hydroxide, preparations in flake form based on silicon dioxide, aluminum oxide or glass which can optionally also be coated with metal oxides, for example titanium dioxide, iron oxides, chromium oxide and chromium hydroxides and mixtures thereof. In that respect the specified synthetic materials in flake form have the advantage that they can be produced in a uniform layer thickness and can already be subjected to further processing more easily at the preliminary supplier.

In accordance with the invention there is provided a composition which can be very easily applied and, as soon as it has been applied, adheres to the position of application for a long time without adversely changing, being transferred onto other articles, bleeding into adjacent regions or running or flaking off. In addition in spite of its low viscosity the composition can also be stored for a very long period of time without constituents settling. The coating or film obtained with the composition according to the invention has the gloss which is desirable for cosmetics.

All these desirable and advantageous properties are achieved if an aqueous composition involves the use of at least one dispersion of a polyurethane polymer in combination with at least one dispersion of an acrylic polymer. For that purpose the dispersions are used in such proportions that it is possible to obtain a preparation having a viscosity in the desired range. Surprisingly it was found that a combination of those two constituents provides that it is possible to form a stable product of low viscosity, the adhesion of which to skin and skin appendages is highly advantageous. Those advantageous properties are achieved only if at least one representative of each of the two polymer classes specified is included. In addition those advantageous properties are achieved without the preparation having to contain a wax. A wax can admittedly be added to that preparation, but it is not necessary to achieve the desired properties and it is therefore not preferred.

As an essential feature of the preparation according to the invention therefore the content of an aqueous dispersion of at least one polycondensate is selected from anion-active, cation-active, non-ionogenic or amphoteric polyurethane polymers or copolymers. In that respect the polyurethane can advantageously be selected from the group of polyester polyurethanes, polyether polyurethanes, polyurethane polyvinyl pyrrolidones, acrylic polyurethanes and silicone-bearing polyurethanes as well as mixtures thereof.

The polyurethane is present in the aqueous phase in the form of particles, the size thereof preferably being in a range of between 10 and 1,000 nm, particularly preferably between 15 and 300 nm and in particular 30 and 100 nm.

All polyurethane dispersions which form a film after application are suitable. Polyurethane polymers of that kind are known to the man skilled in the art. Examples that may be mentioned are polyester polyurethanes as are offered for example by Noveon (formerly BF Goodrich) under the name Avalure, for example Avalure UR-425, Avalure UR-430, Avalure UR-405 and Avalure UR-410, or also products as are furnished by Zeneca under the name Neorez. The preferred polyurethane polymer is one which is identified by the CTFA designation as polyurethane-2.

The polyurethane polymer is used in the form of an aqueous dispersion. Preferably a dispersion of the polymer with a proportion of polymer of between 10 and 80%, preferably between 30 and 60% in water is used. Also suitable are mixtures of the above-listed polyurethane polymers, in particular mixtures of polyester polyurethanes and polyether polyurethanes or also mixtures of polyurethane polymers with polyureas, siliconyl acrylates, PEG/PPG-25/25 and/or dimethicone/acrylates copolymer.

The proportion of the aqueous polyurethane dispersion in the overall composition should be in a range of between 1 and 60%, preferably between 15 and 50%. All percentages in this description, the Examples and the claims always relate to weight unless otherwise stated. In the case of the film which is formed, the polyurethane composition provides inter alia for water-resistance. Below a proportion of 1% the desired effect of the polymer no longer occurs. With more than 60% adjustment of viscosity and the incorporation of coloring agents becomes more difficult.

The second essential component of the composition according to the invention is at least one acrylic-based polymer which provides for adhesion of the composition, gloss and resistance to being rubbed off. In general this involves a polymer which is obtained by radical polymerization and which is preferably an acrylates copolymer or acrylic and/or vinyl polymer and/or acrylic and/or vinyl copolymer. Those polymers are obtained in particular by radical polymerization of suitable monomers, preferably by way of emulsion polymerization. In principle it is also possible for the polymers obtained by radical polymerization to be dissolved in a suitable solvent and for the solution to be re-used. Suitable monomers that may be mentioned are acrylic acid, crotonic acid, maleic acid anhydride or methacrylic acid. Acrylic polymers and copolymers however may also be obtained by polymerization or copolymerization of monomers which are selected for example from monomers such as methyl acrylate, ethyl acrylate, butyl acrylate, methyl methacrylate, ethyl methacrylate, butyl methacrylate, butyl acrylamide, ethyl hexylacrylamide and the like.

Acrylic polymers are also used as aqueous dispersions, the particle size of which is in the same range as in the case of the polyurethane polymer. As the acrylic polymer or acrylates copolymer, it is possible to use both aryl acrylate copolymers and also alkyl acrylate copolymers, wherein in the latter case the alkyl component can have between 1 and 30 C-atoms. The acrylates copolymer is a polymer which is preferably obtained by polarization of at least one monomer which includes an ethylenically unsaturated bond and is selected from α,β-ethylenically unsaturated carboxylic acids, α,β-ethylenically unsaturated carboxylic acid esters and α,β-ethylenically unsaturated carboxylic acid amides and combinations of two or more of those monomers. Preferably acrylic acid, methacrylic acid and esters and amides thereof as well as crotonic acid, maleic acid and itaconic acid are used as the α,β-ethylenically unsaturated carboxylic acids. (Meth)acrylic acid and crotonic acid are preferably used. Particularly preferred are acrylic acid and methacrylic acid and esters thereof.

The α,β-ethylenically unsaturated carboxylic acid esters considered are in particular (meth)acrylic acid esters in which the ester component is a C₁-C₃₀ alkyl residue or C₆-C₁₀ aryl residue. In that case the alkyl and aryl components respectively can be additionally substituted, for example with hydroxy residues. Preferred acrylates which can be used include methyl methacrylate, ethyl methacrylate, butyl methacrylate, isobutyl methacrylate, 2-ethylhexyl methacrylate, lauryl methacrylate and cyclohexyl methacrylate. Preferred aryl methacrylates which can be used include benzyl acrylate and phenyl acrylate. Copolymers of styrene and (meth)acrylic acid (esters) are particularly suitable.

It is moreover also possible to use carboxylic acid amides. Mention may preferably be made of N-alkyl or N-aryl(meth)acrylamides in which the alkyl component can have between 2 and 15 C-atoms or in which the aryl component can have between 6 and 10 C-atoms. N-ethyl acrylamide, N-t-butyl acrylamide, N-t-octyl acrylamide and N-undecyl acrylamide can be preferably mentioned here.

In addition it is also possible to use copolymers of the specified monomers with other olefinically unsaturated monomers. Examples in this respect are acrylic copolymers which were obtained by copolymerization of acrylic acid or methacrylic acid with monomers such as butadiene, styrene or vinyl esters such as vinyl acetate or vinyl benzoate. Preferably copolymers are used which are obtained by copolymerization of acrylic acid or methacrylic acid with styrene or vinyl compounds.

The acrylates copolymer is used in the form of an aqueous dispersion, wherein the aqueous dispersion contains the copolymer in a proportion of between 20 and 70%, particularly preferably between 25 and 40%. The proportion of the acrylates copolymer or the aqueous dispersion of the acrylates copolymer in relation to the overall composition is between 1 and 60%, in each case with respect to the weight of the overall composition, and preferably between 15 and 35%. The desired resistance to being rubbed off is not achieved below a proportion of 1%. When a proportion of more than 60% is involved the swelling capability of the dried film is increased and thus there is a reduction in its water resistance, which leads to poor properties of use. In addition there is a considerable increase in the degree of difficulty in working in coloring agents.

In a preferred embodiment of the invention the acrylic component used is a mixture of at least two different acrylic-based polymers of which one is preferably an acrylates copolymer and the other a copolymer of acrylic acid/methacrylic acid monomers and ethylenically unsaturated aryl monomers. Preferably the latter acrylic polymer is a styrene-bearing acrylates copolymer. In that respect styrene acrylates ammonium methacrylate copolymer is preferably used.

That component is also preferably used in the form of an aqueous dispersion, the dispersion generally containing between 30 and 50% of polymer in water. The proportion of that specific aryl/acrylate copolymer in relation to the overall composition is between 1 and 60%, wherein a proportion in the range of between 15 and 35% and in particular between 20 and 30% is particularly preferred.

In an embodiment which is particularly suitable for application to skin appendages, the aqueous polymer dispersions form between 50 and 90%, preferably between 55 and 75%, of the overall composition. In that case preferably a polyurethane dispersion, an acrylates copolymer dispersion and a styrene/acrylic dispersion are used. That combination is particularly preferably used in such proportions that the polymer proportion is formed for between about 35 and 55% of polyurethane, for between about 15 and 25% of acrylates copolymer dispersion and the balance styrene/acrylic dispersion.

In order to achieve particularly good properties the polymers are preferably so selected that between 30 and 60% of the polymer dispersions of copolymers or polymers which are water-resistant in the dried condition have a glass transition temperature below 10° C. Particularly preferably, with between 5 and 30% of one or more of the dispersions of statistical or block copolymers, a block or portion in the dried condition has a glass transition temperature of more than 30° C.

It was also found that particularly advantageous results are achieved if all film-forming polymers have a minimum film-forming temperature below 30° C. and preferably below ambient temperature.

The preparation according to the invention, in addition to the above-specified polymer components, may contain still further polymers as long as they are compatible and do not adversely affect the properties of the components according to the invention. Thus they may also contain so-called hybrid polymers which can be obtained by copolymerization in the form of acrylic or vinyl polymers with silicone segments or silicone groups. Polycondensates with silicone segments or silicone groups are also known and commercially available.

Polyesters, polyester amides or polyamides are also suitable. Polyesters can be obtained using the methods known to the man skilled in the relevant art, by polycondensation of aliphatic or aromatic dicarboxylic acids with aliphatic or aromatic diols or polyvalent alcohols.

Mention may be made here by way of example of polycondensates comprising adipic acid, succinic acid, glutaric acid, sebacic acid, terephthalic acid or isoterephthalic acid with diols such as ethylene glycol, diethylene glycol, propylene glycol or polyvalent alcohols such as glycerin, trimethylol propane, pentaerythritol, mannitol, sorbitol or xylitol or polymer soft segments such as poly-THF. Polyester amides are obtained in a corresponding manner by the polycondensation of dicarboxylic acids with diamines or amino alcohols.

The following will be mentioned just by way of example as suitable natural polymers: shellac, dammar resin, copal, elemi, tragacanth, gum arabic, xanthane or cellulose derivatives which can optionally be chemically modified, and mixtures thereof.

A further essential feature of the composition according to the invention is viscosity which is in a range of between 0.02 and 3.8 Pa·s, measured at 25° C. and 200 s⁻¹. Viscosity can be measured in per se known manner using a rheometer. The measurement procedure is suitably effected with a rheometer of type BOHLIN CVOR (plate/plate measuring system) at 25° C. and at a shearing rate of 200 s-1 without pre-shearing. In this respect attention is additionally directed to the standard DIN 53018, Part 1 and Part 2.

It was surprisingly found that the composition according to the invention, in spite of the low viscosity, is very durable and can be stored over a long period and by virtue of its structure can also hold particulate constituents in dispersion so that no settlement of the coloring agents or separation into their constituent parts occurs.

Besides the above-described film-forming polymers, depending on the desired properties and the respective area of use, the composition according to the invention may contain further conventional constituents.

If the composition according to the invention is intended for coloring, for example for dyeing hair including eyelashes and/or eyebrows, or however as decorative eye cosmetics, for example as eyeliner, it further contains coloring agents. Coloring agents for compositions of this kind are known per se and those which are suitable for cosmetics can also be used for the material according to the invention.

For coloring the preparation according to the invention it is possible to use inorganic pigments such as titanium dioxide, zinc oxide, iron oxides, chromium oxide green, chromium hydroxide green, ultramarine, carbon black, organic pigments such as carmine and its salts or phthalocyanine, mica, mica coated with metal oxides such as titanium dioxide, iron oxide or chromium oxide, bismuth oxychloride or bismuth oxychloride coated with metal oxides, preparations in flake form based on silicon dioxide, aluminum oxide or glass, which can optionally also be coated with metal oxides, for example titanium dioxide, iron oxides, chromium oxide, chromium hydroxide, metal powders in flake form such as passivated aluminum, bronze, brass, copper, silver, gold, barium, aluminum, strontium, calcium or zirconium lakes of organic dyestuffs or mixtures thereof. When selecting the respectively appropriate coloring agent, consideration should also be given to the national or regional legislation relating to the manufacture of cosmetics as in many countries coloring agents which come into contact with the mucous membranes of the eye must be approved. In Germany for example approval for coloring agents is regulated by Appendix 3 to Regulation 3 of the Cosmetics Regulations.

Both organic soluble dyestuffs and also pigments can be used for coloring keratinic fibers, in particular eyelashes, eyebrows and hair. Pigments are preferred for the composition according to the invention. Both normal pigments and coated pigments can be used. The pigments are preferably of a small particle size, preferably in a range of between 20 nm and 150 μm. For that purpose the pigments are finely ground in the usual manner or possibly used in micronized form.

If the composition according to the invention is to be used for coloring eyelashes and eyebrows, for example black iron oxide or ultramarine blue are suitable coloring agents as pigments. The proportion of the pigment depends on the desired coloration and suitable amounts are known per se to the man skilled in the art. For example a proportion of between 0 and 30%, in particular between 5 and 25%, is to be considered for the composition according to the invention. For specific dyestuffs with which particular effects are to be achieved however proportions outside those ranges are optionally also possible.

The storability of the products according to the invention can be improved by using preferably pigments with a hydrophobic, physically or chemically adhering coating. Coatings for pigments are known per se and do not need to be described in greater detail here. Suitable examples are silicone-bearing coatings with dimethicone or other silicones or coatings with titanium triisostearate. The use of coated pigments affords the further advantage that wetting or swelling of the film formed, upon contact with water, is minimized. That further enhances the durability and resistance of the film formed. Even if the film swells somewhat upon coming into contact with water, it remains completely intact and stable if it is not exposed to mechanical friction in the swollen condition.

When using strongly hydrophobically coated pigments it is recommended that a surface-active substance also be added to the composition, optionally in combination with a polymeric wetting agent. Such surface-active substances and wetting agents contribute to the water compatibility of the particles.

Examples of suitable surface-active agents that may be mentioned are low-molecular tensides such as sarcosinates, non-ionic and/or anion-active tensides such as PEG-modified fatty acids and fatty alcohols, sodium laureth sulfate, solubilizers as are also conventional for the compatibilisation of UV substances, and/or ester oils.

If they are present the low-molecular tensides are used preferably in a proportion of between 0.1 and 3% in each case with respect to the weight of the overall composition.

Suitable polymeric wetting agents are for example silicone tensides and amphiphilic siliconyl acrylates and PVP derivatives, for example VP/eicosene and VP/hexadecane copolymer, fluorotensides, cholesterol esters, lecithin and acrylate copolymers, for example an acrylate block copolymer and other known polymer tensides. An acryl-bearing wetting agent is particularly preferably used as the polymeric wetting agent in order to improve compatibility. If present the polymeric wetting agent is appropriately used in a proportion of between 0.05 and 5%, preferably between 0.1 and 3%. In a preferred embodiment a non-ionogenic wetting agent, in particular a dimethicone copolyol or a non-ionogenic polymeric wetting agent such as for example an acrylate or polyethylene block copolymer is used in a small amount, preferably between 0.05 and 3% by weight, quite particularly preferably between 0.3 and 1.5% by weight. That provides that the coloring agent particles on the one hand are well wetted and on the other hand are bound into the polymer structure by molecular interactions. At the same time that provides that, upon drying, the film is particularly smooth and thereby affords a high level of surface gloss. That can also be achieved by the use of polymer segments with a reflection capability which is known to be good, and with a relatively high glass transition temperature T_{g}, such as for example polystyrene.

In a preferred embodiment of the preparation according to the invention which is pigmented preferably thickening agent which provides for still better storage stability is used together with the pigments. As at the same time however the material obtained should not become crumbly but must be such that it can be applied smoothly, not all known thickeners are suitable for that purpose. In particular pyrogenic silicic acid is unsuitable as it results in the composition crumbling away after it has been applied. In addition the preparation according to the invention should be of such a low viscosity that it is free-flowing and therefore can also be stored in vessels of the most widely varying geometry without a problem arising in terms of removal therefrom. It was surprisingly found that a low-viscosity preparation can be obtained if a thickener mixture is used, which contains at least one inorganic laminate silicate combined with a xanthane gum. Suitable mixtures are those comprising natural and synthetic as well as modified polysaccharides, mineral and modified mineral thickeners, silicate thickeners, celluloses and modified celluloses, non-ionic associative thickeners, copolymer thickeners such as hydroxyethyl acrylate/sodium acryloyl dimethyltaurate copolymer, sodium acrylate acryloyl dimethyltaurate copolymer, sodium acrylate/acryloyl dimethyltaurate copolymer, and VP/dimethiconyl acrylate/polycarbamoyl/polyglycol ester. Particularly good properties are achieved if a mixture is used comprising a larger proportion of a hydrocolloid or vegetable gum, for example xanthane gum, a smaller proportion of a silicatic thickener and a small proportion of a polysaccharide, in particular cellulose, which can optionally also be chemically modified. By way of example reference may be made to a mixture comprising between 40 and 70% xanthane gum, between 10 and 40% of mineral thickener and the balance cellulose or cellulose derivative. The thickener used is particularly preferably a mixture of cellulose, silicate and xanthane gum in a ratio in the range of between 1:1:4 and 1:2:6.

The thickener mixture can be added in a proportion of up to 20%, depending on the respectively desired viscosity. In general terms proportions in the range of between 0.2 and 5% have proven to be suitable, in each case in relation to the weight of the overall composition. If a lower viscosity is wanted it will be appreciated that the thickener proportion can be used in the lower range while the proportion of thickener is increased if the viscosity is to be increased.

The thickener mixture used according to the invention stabilizes the pigments and thus substantially prevents the pigments from settling. In that way the pigments remain homogenously distributed even when stored over a relatively long period of time and do not have to be re-dispersed by being shaken up prior to each use.

On the other hand the low viscosity which can be achieved in spite of the use of the thickener mixture provides that the material is not applied on the eyelash and nonetheless provides for good coloration thereof. A disadvantage with a relatively thick application is that in that situation, when drying occurs, stresses are produced which have the result that the material flakes off after some time. In contrast the composition according to the invention provides a coating which remains durably at the location of application for a long period of time, by virtue of its elasticity and its uniform layer thickness.

In an embodiment the preparation according to the invention may also contain one or more conventional and per se known sun protection agents or UV-absorbent agents in order to protect skin or hair from solar radiation. Frequently this embodiment is used without a pigment addition. The sun protection agents are used in the amounts which are conventional for cosmetics.

If a preservation effect is wanted for the composition according to the invention, it is possible for that purpose to use all common preserving agents, inter alia carbamates, for example iodine propinyl butyl carbamate. The use of those preserving agents is implemented up to the amounts which are allowed by statute, as are set out for example in Germany by Appendix 6 to Regulation 3a of the Cosmetics Regulations in the respectively applicable wording.

In order further to improve the rheological properties of the composition according to the invention and application of the composition it is optionally also possible to use co-solvents, that is to say water-miscible solvents. Suitable substances here are for example monovalent and/or polyvalent alcohols with between 2 and 20 carbon atoms, esters and ethers thereof, liquid compounds which are miscible with water or which are easily dispersible in water such as for example N-methyl pyrrolidone, dimethyl acetamide, glycerin, diglycerin, triglycerin, butylene glycol, hexane triol, propylene glycol, dipropylene glycol, hexylene glycols, mixtures of dimethicone copolymer and caprylic/capric triglyceride or other suitable silicone tensides, as well as solutions of sold substances which are easily soluble in water and which serve as agents for keeping the material moist but not as co-solvents, such as for example urea, xylitol, inositol, maltitol, sorbitol, mannitol, glucose, fructose, sucrose, lactose, trehalose, diglycet-7-malate, glyceryl dimaltodextrin, sorbityl silanediol, as well as amino acids or nitrogen-bearing compounds such as acetyl arginine, amidinoproline, betaine, chitosan lauroyl glycinate, diglycol guanidine succinate, derivatives of guanidine acetate and mixtures thereof. Preferably the co-solvents used are polyvalent alcohols which at the same time also serve as agents for keeping the material moist. The co-solvent is usually contained in the material in a proportion of between 0 and 5%.

The film-forming polymers used according to the invention have a glass transition temperature in a range which causes a soft and elastic film to be produced, even without a plasticiser. If however the softness and elasticity are to be increased still further or if running of the material is to be further promoted, it is also possible to add a plasticiser which is known for cosmetic materials. Suitable materials in this respect are both low-molecular and also higher-molecular plasticisers which are optionally used solubilized or dissolved in a co-solvent. Silicone tensides, but also short-chain ester oils, are considered as being suitable here. The hardness of the film formed from the preparation according to the invention can also be influenced by the addition of benzoyl benzoate, tributyl citrate, trihydroxy propyl citrate, or laureth-2-benzoate.

If present the plasticisers can be used in per se known manner, for example in a proportion of between 0 and 5%.

In order to prevent foaming of the composition during manufacture or in use—for example in the case of the eyebrow mascara due to the “air pumping effect” when the applicator is pulled out of and re-introduced into the container—, which can be disadvantageous when applying the composition, it is possible to add per se known anti-foam agents in a small amount. Examples in that respect are silicone compounds as are also used for other cosmetic compositions. Usually a silicone compound of that kind is used in the form of an emulsion in a proportion of between about 0 and 5%, preferably between 0.1 and 1%.

When the applied material is to be removed again that can be effected in a simple manner by water being applied to the applied film at relatively high temperature, for example a temperature above 25° C., preferably between 30 and 38° C., and by the film then being removed by mechanically rubbing, for example with a cotton wool pad.

The preparation according to the invention can be produced in per se known manner. In order to obtain a particularly smooth and homogenous material which can be well applied, production is preferably effected by a procedure whereby firstly the polymer dispersions are prepared and heated to an elevated temperature, preferably in the range of between 45 and 60° C.

If a thickener mixture is used, it is preferably pre-dispersed in a co-solvent which can be mixed in any desired manner with water but which is different from water, for example a polyvalent alcohol, glycerin or ester oil. Both mixtures are then mixed together and the further constituents are added in the usual manner. The coloring agents can then be worked into the homogenous dispersion and uniformly distributed therein.

That material can now be readily introduced into the device from which the composition is to be delivered. The composition according to the invention is so stable that, even without agitation, it does not settle or separate into its constituent parts. The composition can possibly also be introduced while still hot immediately after manufacture, into the containers provided for it.

In rare cases, during the cooling operation, some water may be deposited at the surface, which however can be homogenously distributed again by simply stirring it in. As the viscosity of the composition according to the invention is low, it can also be filled into the containers intended for it in the cold condition, which is advantageous in terms of preserving the structure of the material.

If desired, after completion of the composition, adjuvants such as plasticisers, gloss-imparting agents, run adjuvants and so forth can also be added to the composition.

The composition according to the invention can be used for the treatment of skin and skin appendages. In particular the composition according to the invention is suitable on the one hand for making up the eyes and on the other hand for the treatment of keratinic material, in particular eyelashes, eyebrows and hair.

A further subject of the invention is therefore use of the preparation according to the invention for making up eyes, preferably the eyelids, eyelid edges, eyelashes and eyebrows.

A further subject of the invention is use of the preparation according to the invention for the treatment of keratinic material, in particular hair, eyelashes and eyebrows, both in the natural condition and also the synthetic imitations thereof.

Preferably the preparation according to the invention is used to provide keratinic material with a coating, in particular a coloring coating. That coating is durable over the long term and is maintained even upon coming into contact with water. The coloring is maintained for up to 4 days. If desired the coating can be removed again at any time by applying water, preferably at relatively high temperature, and removing the coating by mechanical rubbing after a few minutes.

If the preparation according to the invention is used for application to keratinic material, the application can preferably be implemented with an applicator system which includes a container and a removable element for closure thereof, wherein the closure element is preferably an application element, that is to say an applicator.

A further subject of the invention is therefore also a device for applying a preparation, as described hereinbefore, which includes a container which can accommodate the preparation and a neck portion with a screwthread and seal, and an applicator element which at its one end forms a brush and at its other end a handle forming a closure cap, wherein the brush head is of a diameter in the range of between 5.5 and 6.5 mm and a length in the range of between 12.5 and 17.5 mm and wherein the bristles are arranged in a helical arrangement radially around a central portion with a high number of turns.

In a preferred embodiment the applicator system has a container which contains the preparation according to the invention and which is provided with a neck portion. The neck portion has an annular stripper or wiper. The element for closing the container carries an applicator which has a stem and which at its first end is provided with a brush as the applicator element and which at its second end is connected to a handle. In the closed condition the closure element closes the container, in which case the applicator mounted on the closure element dips into the preparation. To apply the preparation the closure element is pulled out, in which case the applicator moves over the annular wiper and in that operation wipes off excess material. The preparation can then be applied with the brush. As the preparation according to the invention is of an extremely low viscosity, it is possible for the container to be designed in many different geometries, which is not possible with the known applicator systems for mascara. Conventional mascara is of a relatively high viscosity and therefore can only be stored in cylindrical containers into which the applicator can be introduced in such a way that it can remove the material. As the preparation according to the invention is of such a viscosity that the material is freely flowing, the preparation can also be introduced into more bulbous or rounded vessels or vessels of any geometry.

In a preferred embodiment the applicator for applying the preparation satisfies the following conditions so that application is homogenous and resistant. In the preferred embodiment the applicator is a brush with soft bristles so that, upon repeated application, the bristles cannot damage the layer which has already been applied and thereby make it open to attack by water. In addition the brush is briefly of large diameter so that individual parts of eyebrows can be specifically colored. In a particularly preferred feature the brush used is of a diameter at the tip of between 5.5 and 6.5 mm, a length of between 12.5 and 17.5 mm with nylon as the fiber material measuring 2.5 mils. Particularly preferably a brush with a large number of turns is used.

In accordance with the invention there is provided a preparation which can be applied durably and resistantly to skin and skin appendages and which can remain thereon for a very long period of time. As soon as the preparation has been applied to the desired part and has dried, which requires only a short period of time, it remains adhering to the location of application without transferring onto articles which come into contact therewith, without bleeding into near regions or crumbling off. The preparation is therefore ideally suited for application to keratinic material, in particular eyelashes, eyebrows and hair. The preparation can be applied to hair in order to impart thereto a protective coating and at the same time body and life. In a preferred embodiment the preparation is provided with coloring agents and serves for coloring the keratinic material.

The invention will be further described by means of the following Examples without being restricted thereto.

### EXAMPLE 1

### Body Tattoo

| | % by wt. |
|---|---|
| Isodecane acrylates copolymer | 23.500 |
| Aqua | 19.400 |
| PPG-17/IPDI/DPMA copolymer | 20.000 |
| Acrylates copolymer | 16.500 |
| Propylene glycol | 3.500 |
| Methyl eugenol PEG-8 dimethicone | 2.200 |
| Adipic acid/diethylene glycol glycerin crosspolymer | 2.000 |
| C12/C15 alkyl benzoate | 1.000 |
| Caprylic/capric triglyceride | 1.000 |
| Preservatives | 0.750 |
| Dimethicone | 0.150 |
| Pigments | 10.000 |

### EXAMPLE 2

### Lipliner

| | % by wt. |
|---|---|
| Aqua | 43.750 |
| Polystyrene | 16.000 |
| Polyurethane-4 | 15.000 |
| Glycerin | 6.000 |
| Acrylates copolymer | 4.000 |
| Caprylic/capric triglyceride | 3.000 |
| PEG-150/decyl alcohol/SMDI copolymer | 1.000 |
| Preservatives | 0.750 |
| Dimethicone | 0.500 |
| Pigments | 10.000 |

### EXAMPLE 3

### Eyeliner

| | % by wt. |
|---|---|
| Acrylates copolymer | 30.000 |
| Aqua | 24.450 |
| Polystyrene | 18.000 |
| Caprylic/capric triglyceride | 5.000 |
| Propylene glycol | 2.500 |
| Sorbitol | 1.500 |
| Acetyl tributyl citrate | 2.000 |
| Preservatives | 0.750 |
| Sodium cocoamphoacetate | 0.500 |
| PVP/dimethiconyl polycarbamyl/polyglycerol ester | 0.300 |
| Pigments | 15.000 |

### EXAMPLE 4

### Lip Rouge

| | % by wt. |
|---|---|
| Aqua | 44.250 |
| Ammonium acrylates copolymer | 15.000 |
| Polyurethane-1 | 10.000 |
| Dimethicone copolyol | 6.000 |
| Adipic acid/diethylene glycol/glycerin crosspolymer | 5.000 |
| Caprylic/capric triglyceride | 4.000 |
| Sodium cocoamphoacetate | 1.000 |
| PEG-180/laureth-50/TMMG copolymer | 1.000 |
| Dimethicone | 1.000 |
| Preservatives | 0.750 |
| Pigments | 12.000 |

### EXAMPLE 5

### Eye-Shadow (Liquid)

| | % by wt. |
|---|---|
| Aqua | 32.750 |
| Ammonium acrylates copolymer | 15.000 |
| Polyurethane-1 | 10.000 |
| Adipic acid/diethylene glycol/glycerin crosspolymer | 5.000 |
| Dimethicone copolyol | 4.500 |
| Caprylic/capric triglyceride | 4.000 |
| Propylene glycol | 3.000 |
| Sorbitol | 1.500 |
| Sodium cocoamphoacetate | 1.000 |
| PEG-180/laureth-50/TMMG copolymer | 1.000 |
| Preservatives | 0.750 |
| Dimethicone | 0.500 |
| Pigments | 21.000 |

### COMPARATIVE EXAMPLE 6

### Eyeliner

| | % by wt. |
|---|---|
| Polyurethane-7 | 86.250 |
| Dimethicone copolyol | 4.000 |
| Caprylic/capric triglyceride | 3.500 |
| Glycerin | 3.000 |
| Preservatives | 0.750 |
| Dimethicone | 0.500 |
| Pigments | 2.000 |

A preparation produced in accordance with the Comparative Example—which is not in accordance with the invention—has too little covering power, applies irregularly and fails in the preservation loading test—in spite of an apparently sufficient amount of preserving agent which corresponds to the content in the Examples according to the invention.

### EXAMPLE 7

### Eyelash Mascara

| | % by wt. |
|---|---|
| Water | q.s. 100 |
| Dicaprylyl maleate | 0.50 |
| Preserving agent | 0.60 |
| Hydrophobically modified pigment | 10.0 |
| VP/hexadecane copolymer | 0.30 |
| Sorbitol | 1.50 |
| Acrylates copolymer | 20.00 |
| Styrene/acrylates copolymer | 12.00 |
| Polyurethane-2 | 28.00 |
| Caprylic/capric triglyceride | 1.00 |
| Trimethyl siloxyamodimethicone | 1.00 |
| Anti-foaming agent emulsion | 0.40 |
| Butane-1,4-diol | 3.00 |
| Xanthane gum | 0.40 |
| Bentonite | 0.25 |
| Gellan gum | 0.08 |

### EXAMPLE 8

### Eyebrow Mascara

| | % wt. by |
|---|---|
| | q.s. 100 |
| Laureth-2-benzoate | 0.70 |
| Preserving agent | 0.70 |
| Hydrophobically modified pigment | 8.00 |
| Hydrolized wheat protein/dimethicone copolyol acetate | 0.40 |
| Sorbitol (aqueous solution) | 1.50 |
| Styrene/VP-copolymer (aqueous solution) | 6.00 |
| Ammonium acrylates copolymer (aqueous solution) | 25.00 |
| PPG 17/IPDI/DMPA-copolymer (aqueous solution) | 30.00 |
| Caprylic/capric triglyceride | 1.00 |
| Glycerin | 1.00 |
| Bis-PEG/PPG-20/20 dimethicone | 1.00 |
| Anti-foaming agent (aqueous emulsion) | 0.40 |
| Propane-1,3-diol | 3.50 |
| Hydroxypropyl cellulose | 0.11 |
| Xanthane gum | 0.30 |
| Hectorite | 0.15 |

The foregoing constituents are used to produce an eyebrow mascara by a procedure whereby the acrylate copolymer dispersion and the polyurethane dispersion are provided and preheated to between 50 and 55° C. and then the further ingredients are added. The mixture is stirred until there is homogenous material. The material can then be introduced into a container for accommodating it, as it is or in a cooled condition.

When that material is applied to eyelashes, the result produced is a smooth colored coating which adheres to the eyelash for a long time.

For compositions which were produced as described hereinbefore viscosity was determined as follows:

Measuring apparatus: Bohlin CVOR Rheometer,

Shearing stress ramp 1 to 1000 Pa upwards for recording a flow limit

Measuring system: plate/plate 20 to 0.4 mm gap spacing

Temperature: 25° C.

Measurement Without Pre-Shearing

Measuring time: 120 s plus 60 s pre-heat treatment at 25° C.

Conversion to a viscosity curve is effected electronically

For compositions produced in accordance with the invention, viscosities were measured under those conditions in the range below 2,500 mPa.s.

1-24. (canceled)

25. An aqueous-based preparation for application to the skin or skin appendages comprises a mixture of at least two aqueous dispersions of film-forming polymers, wherein one dispersion is a polyurethane-based polymer and the other dispersion is an acrylic-based polymer, wherein the preparation has a viscosity in the range of between 0.02 and 3.8 Pa·s measured at 25° C. and with 200 s⁻¹.

26. A preparation as set forth in claim 25, wherein the polyurethane-based polymer comprises at least one polymer is selected from the group consisting of polyester polyurethanes, polyether polyurethanes, polyurethane polyvinyl pyrrolidones, acrylic polyurethanes, silicone-bearing polyurethanes and mixtures thereof.

27. A preparation as set forth in claim 25, wherein the aqueous dispersions form between 50 and 90% by wt. with respect to the wt. of the final preparation.

28. A preparation as set forth in claim 26, wherein the polyurethane-based polymer is present in an amount of between 1 and 60% by wt. with respect to the weight of the final preparation.

29. A preparation as set forth in claim 25, wherein the acrylic-based polymer comprises an acrylic polymer obtained by polymerization of at least one monomer which contains an ethylenically unsaturated bond, selected from the group consisting of α,β-ethylenically unsaturated carboxylic acids, α,β-ethylenically unsaturated carboxylic acid esters, α,β-ethylenically unsaturated carboxylic acid amides, and mixtures thereof.

30. A preparation as set forth in claim 25, wherein the acrylic-based polymer is obtained by polymerization of monomers selected from the group consisting of acrylic acid, methacrylic acid, crotonic acid, maleic acid, itaconic acid, and mixtures thereof.

31. A composition as set forth in claim 25, wherein the acrylic-based polymer is selected from aryl and alkyl acrylates and mixtures thereof.

32. A preparation as set forth in claim 31, wherein the acrylic-based polymer comprises a mixture of acrylates copolymer and styrene/acrylic copolymers.

33. A preparation as set forth in claim 25, wherein the polymers are present in the dispersions in the form of particles of a size of between 10 and 1000 nm.

34. A preparation as set forth in claim 25, wherein the polymers in the dispersions have film-forming temperatures of below 30° C.

35. A preparation as set forth in claim 25, wherein at least 30% by wt. of the polymers which are contained in the dispersions are water-resistant in the dried condition and have a glass transition temperature of below 10° C.

36. A preparation as set forth in claim 25, wherein between 5 and 30% by wt. of one or more of the dispersions have a block or portion whose glass transition temperature in the dried condition is higher than 30° C.

37. A preparation as set forth in claim 25, further comprising, conventional constituents selected from the group of plasticisers, wetting agents, anti-foaming agents, coloring agents, preserving agents, fragrances, flavoring agents and mixtures thereof.

38. A preparation as set forth claim 30, further comprising at least one pigment.

39. A preparation as set forth in claim 30, further comprising at least one thickener.

40. A preparation as set forth in claim 39, wherein the thickener is selected from the group consisting of natural and/or synthetic and/or modified polysaccharides, mineral and/or modified mineral thickeners, silicate thickeners, celluloses and modified celluloses, non-ionic associative thickeners and/or copolymer thickeners and mixtures thereof.

41. A preparation as set forth in claim 25, further comprising at least one polysaccharide, at least one mineral thickener and at least one vegetable gum.

42. A preparation as set forth in claim 39, wherein the thickener is selected from the group consisting of cellulose, a cellulose derivative, a silicatic thickener, xanthane gum and mixtures thereof.

43. A preparation as set forth in claim 42, wherein cellulose, silicate and xanthane gum are present in a ratio of between 1:1:4 and 1:2:6.

44. A preparation as set forth in claim 39, wherein the thickener is present in an amount of between 0.2 and 20% by wt. with respect to the weight of the final composition.

45. A preparation as set forth in claim 39, wherein the thickener is present in an amount of between 0.2 and 5% by wt. with respect to the weight of the final composition.

46. A preparation as set forth in claim 25, comprising a keratinic material.

47. A preparation as set forth in claim 45, for application to hair, eyelashes and/or eyebrows.

48. A preparation as set forth in claim 45, comprises make up for the eyes and eyelashes.

49. A device for applying a preparation as set forth in claim 25, comprise a container which can accommodate the preparation and which has a neck portion with a screwthread and a seal, and an applicator element which at its one end has a brush and at its other end a handle which forms a closure cap, wherein the brush head is of a diameter in the range of between 5.5 and 6.5 mm and is of a length in the range of between 12.5 and 17.5 mm and wherein the bristles are arranged in a helical arrangement radially around a central portion with a large number of turns.
